(19) United States
(12) Patent Application Publication
(10) Pub. No.: US 2005/0150715 A1
(43) Pub. Date: Jul. 14, 2005

(54) Hanging sound suppression device

(76) Inventor: Richard Douglas Barnes, Flat Top, WV (US)

Correspondence Address: WEISS & MOY PC, 4204 NORTH BROWN AVENUE, SCOTTSDALE, AZ, 85251, US

(21) Appl. No.: 10/755,554

(22) Filed: Jan. 12, 2004

(51) Int. Cl. H04R 25/02; A61B 7/02

(52) U.S. Cl. 181/135; 181/130

(57) ABSTRACT

A hanging sound suppression device consists of a sound suppression device coupled via a connector to a wearable object. In one embodiment, the wearable object is an earring. In another, it is an eyeglass frame. When not in use, the sound suppression device is permitted to dangle from the wearable object.

### FIELD OF THE INVENTION

This invention relates generally to sound suppression and, more particularly, to a hanging sound suppression device, in which a sound suppression device such as an earplug is coupled to an earring or eyeglass frame and thereby located in proximity to a user's ear and available for use.

### BACKGROUND OF THE INVENTION

The need for sound suppression devices, including particularly ear plugs, arises in a variety of contexts. For example, persons observing a NASCAR® race, drag race, or the like often desire to use earplugs, for protection against the loud sound produced by the car engines. Those engaged in recreational shooting activities have a similar need for sound protection. Myriad other circumstances call for the use of sound protection devices.

It is often the case that persons who need sound protection find that they have either forgotten to bring ear plugs and/or that they cannot purchase them when they need them. Moreover, even where available, typical prior art earplugs are relatively unattractive.

A need therefore existed for an earplug that would be more easily carried by a user, and therefore more available when needed. Preferably, such an earplug should also be more attractive in appearance than prior art devices. The present invention satisfies these needs and provides other, related, advantages.

### SUMMARY OF THE INVENTION

In accordance with one embodiment of the present invention, a hanging sound suppression device is disclosed. The device comprises, in combination: an object worn by a user; a sound suppression device; and a connector coupled at a first end thereof to the object and at a second end thereof to the sound suppression device.

### BRIEF DESCRIPTION OF THE DRAWINGS

FIG. 1 is a perspective view of an embodiment of a hanging sound suppression device, consistent with the present invention, in which the sound suppression device is coupled to an earring that is in position on the ear of a wearer.

FIG. 2 is a perspective view of an embodiment of a hanging sound suppression device and earring, consistent with the present invention, in which the sound suppression device is coupled to an earring.

FIG. 3 is a perspective view of an embodiment of a hanging sound suppression device, consistent with the present invention, in which the sound suppression device is coupled to an earring.

FIG. 4 is a front view of the sound suppression device portion of an embodiment of a hanging sound suppression device consistent with the present invention.

FIG. 5 is a perspective view of a hanging sound suppression device, consistent with an embodiment of the present invention, coupled to an eyeglass frame.

FIG. 6 is a perspective view of the embodiment of FIG. 5, illustrating placement of the sound suppression device in the ear of a user.

### DETAILED DESCRIPTION OF THE PREFERRED EMBODIMENTS

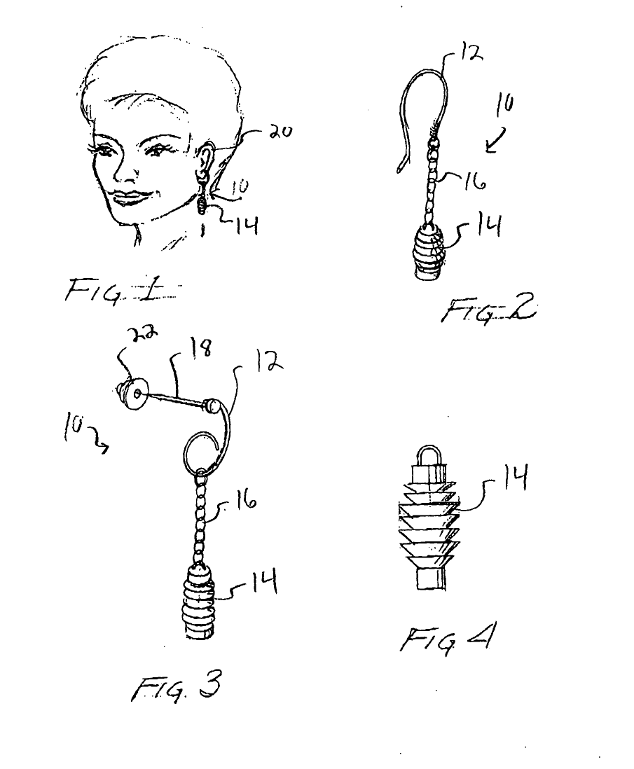

Referring first to FIGS. 1-3, an embodiment of a hanging sound suppression device 10 (hereinafter “device 10”) consistent with the present invention is shown. In this embodiment, the device 10 comprises an earring 12, a sound suppression device 14, and a connector 16 connecting the suppression device 14 to the earring 12.

Referring first to FIG. 3, in one embodiment, the earring 12 is a post type of earring, having a post 18 secured in position through an ear 20 (see FIG. 1) with a backing 22. FIG. 2 is an example of an earring 12 that is hook-type. It should be noted that the earring 12 could also be of a clip-on variety.

With respect to the sound suppression device 14, this may be constructed of any suitable sound suppression material, such as foam. It is preferred to provide a sound suppression device 14 having an aesthetically pleasing shape. In this fashion, the device 10 optimally has an appearance of a dangling earring, without the sound suppression function being unduly apparent. The shape of the sound suppression device 14 may be thematically connected to the event at which the sound suppression device 14 is to be used. For example, where it is to be used at a NASCAR® race, it may be desired to provide a sound suppression device 14 having the shape of a race car.

The connector 16 couples the sound suppression device 14 to the earring 12. It is preferably chain type, as shown by way of example in FIGS. 2 and 3, though other forms of connectors may also be utilized. The purpose of the connector 16 is to permit the sound suppression device 14 to dangle outside of the ear 20 until it is desired to be used. When needed, the user can grasp the sound suppression device 14 and insert it into its proximate ear 20. The ends of the connector 16 may be secured to the earring 12 and sound suppression device 14 in any desired manner. For example, the end of the connector 16 proximate the earring 12 may be welded thereto. The end of the connector 16 proximate the sound suppression device 14 may be inserted therein (as shown by way of example in FIGS. 3 and 4), glued thereto, or otherwise affixed.

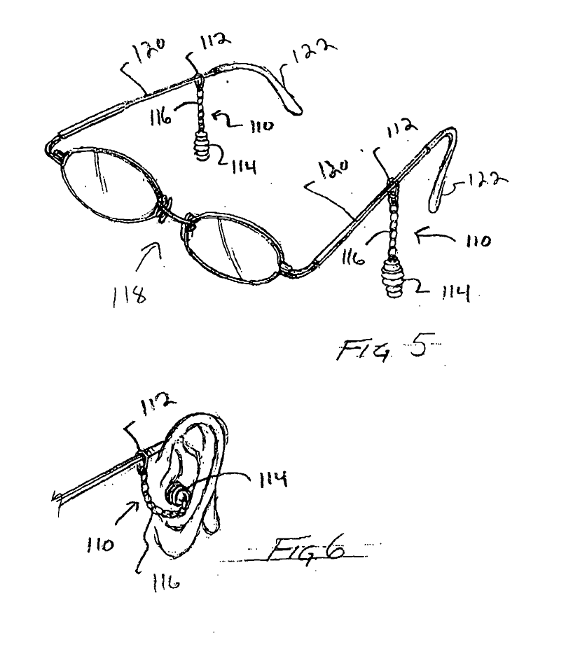

Referring now to FIGS. 5-6, another embodiment of a hanging sound suppression device (hereinafter “device 100”) consistent with the present invention is shown. In this embodiment, the device 100 comprises an eyeglass frame clasp 112 (“clasp 112”), a sound suppression device 114, and a connector 116 connecting the suppression device 114 to the clasp 112.

The clasp 112 should be adapted to couple to a portion of the frame of a pair of eyeglasses 118. By way of example, as shown in FIGS. 5 and 6, coupling of each clasp 112 could be to the temple 120 portion. The clasp 112 should be dimensioned so as to facilitate such coupling. It may have a loop shape, as shown by way of example in FIGS. 5 and 6, so that it may be slid over an end of the ear piece 122 portion of the eyeglasses 118. Other configurations may also be desired.

With respect to the sound suppression device 114, reference is made to the description provided above concerning the sound suppression device 14. With respect to the connector 116, reference is made to the description provided above concerning the connector 16.

The embodiment of FIGS. 5 and 6, it should be noted, may be particularly desired for users engaged in shooting activities, where the eyeglasses 118 serve as eye protection and the sound suppression devices 114 provide hearing protection.

It should be noted that eyeglasses 118 and earrings 12 represent examples of objects that are wearable by a user and to which a sound suppression device may be coupled. Other objects may be utilized as well, including for example a hat.

While the invention has been particularly shown and described with reference to preferred embodiments thereof, it will be understood by those skilled in the art that the foregoing and other changes in form and details may be made therein without departing from the spirit and scope of the invention. For example, with respect to the embodiment of FIGS. 1-4, it may be desired to provide a combination earring and sound suppression device in which the sound suppression device is removably coupled directly to the earring. In such a configuration, the sound suppression device could be grasped, removed from the earring when needed, and inserted into the ear. After use, the sound suppression device could be removed and re-attached to the earring. Other variations on the basic concept may also be possible without departing from the spirit or scope of the present invention.

1. A hanging sound suppression device comprising, in combination:
an object worn by a user;
a sound suppression device; and
a connector coupled at a first end thereof to said object and at a second end thereof to said sound suppression device.

2. The device of claim 1 wherein was said object is an earring.

3. The device of claim 2 wherein said earring is post-type.

4. The device of claim 1 wherein said connector comprises a chain.

5. The device of claim 2 wherein said sound suppression device is permitted to dangle below said earring when said sound suppression device is not in a user's ear.

6. The device of claim 1 wherein said object is a portion of an eyeglass frame.

7. The device of claim 6 wherein said portion is a temple.
